(19) United States
(12) Patent Application Publication
(10) Pub. No.: US 2026/0262972 A1
(43) Pub. Date: Sep. 10, 2026

(54) SIGNAL PROCESSING DEVICE, MEDICAL PHOTOMETER, NON-TRANSITORY COMPUTER-READABLE MEDIUM, AND PHOTOMETRY METHOD

(71) Applicant: NIHON KOHDEN CORPORATION, Tokyo (JP)

(72) Inventor: Yoshinori Ueda, Saitama (JP)

(73) Assignee: NIHON KOHDEN CORPORATION, Tokyo (JP)

(21) Appl. No.: 19/560,558

(22) Filed: Mar. 9, 2026

(30) Foreign Application Priority Data
Mar. 10, 2025 (JP) 2025-037642

(51) Int. Cl. A61B 5/1455; A61B 5/00

(52) U.S. Cl. CPC A61B 5/14552; A61B 5/7278; A61B 2562/0238

(57) ABSTRACT

An interface receives first, second, and third signals respectively corresponding to intensities of first, second, and third light respectively including first, second, and third wavelengths that have passed through a tissue of a subject. A filter allows passage of a signal having a frequency included in a specific frequency band. A processor calculates a concentration of a light absorber in blood of the subject based on the first and the second signals that have passed through the filter. A ratio of extinction coefficients of a first and second light absorbers in blood is different between the first and second wavelengths. Any one of the first and second light absorbers in blood is the light absorber in blood. The third signal has a larger amplitude than the first and second signals. The processor determines the frequency band based on a frequency of the third signal.

### CROSS-REFERENCE TO RELATED APPLICATION

The present application is based on Japanese Patent Application No. 2025-037642 filed on Mar. 10, 2025, the entire contents of which are incorporated herein by reference.

### BACKGROUND

The present disclosure relates to a device configured to process a signal corresponding to an intensity of light that has passed through a tissue of a subject. The present disclosure also relates to a medical photometer including the device and a sensor configured to output the signal. The present disclosure also relates to a non-transitory computer readable medium having stored a computer program adapted to be executed by a processor installed in the device. The present disclosure also relates to a photometry method adapted to be performed with the device.

A pulse photometer as an exemplary medical photometer is a device configured to perform, as an exemplary photometry method, calculation of a light absorber concentration in blood of a subject. Specifically, light beams with different wavelengths are irradiated onto a tissue of the subject. The wavelengths are so determined that a ratio of blood extinction coefficients varies in accordance with the light absorber concentration in blood. A quantity of light of each wavelength that has passed through the tissue is detected. The quantity of light of each wavelength varies in accordance with blood pulsations of the subject. Accordingly, a pulse wave signal representing temporal variations of the light quantity of each wavelength caused by the pulsations is obtained. An amplitude of the pulse wave signal associated with each wavelength corresponds to a variation of light attenuation for the wavelength. The light absorber concentration in blood is calculated based on a ratio of the variations of light attenuations for the respective wavelengths (see, for example, Japanese Patent No. 4196209B).

### SUMMARY

It is required to suppress a decrease in calculation accuracy of the light absorber concentration in blood even in a case where the amplitude of the pulse wave signal to be obtained is small.

An illustrative aspect of the present disclosure may provide a signal processing device, comprising:
an interface configured to receive:
   a first signal corresponding to an intensity of first light that has passed through a tissue of a subject, the first light including a first wavelength;
   a second signal corresponding to an intensity of second light that has passed through the tissue, the second light including a second wavelength; and
   a third signal corresponding to an intensity of third light that has passed through the tissue, the third light including a third wavelength;
a filter configured to allow passage of a signal having a frequency included in a specific frequency band; and
a processor configured to calculate a concentration of a light absorber in blood of the subject based on the first signal and the second signal that have passed through the filter,
wherein a ratio of an extinction coefficient of a first light absorber in blood and an extinction coefficient of a second light absorber in blood is different between the first wavelength and the second wavelength;
wherein any one of the first light absorber in blood and the second light absorber in blood is the light absorber in blood;
wherein the third signal has a larger amplitude than the first signal and the second signal; and
wherein the processor is configured to determine the frequency band based on a frequency of the third signal.

An illustrative aspect of the present disclosure may provide a medical photometer, comprising:
a sensor configured to output:
   a first signal corresponding to an intensity of first light that has passed through a tissue of a subject, the first light including a first wavelength;
   a second signal corresponding to an intensity of second light that has passed through the tissue, the second light including a second wavelength; and
   a third signal corresponding to an intensity of third light that has passed through the tissue, the third light including a third wavelength; and
a signal processing device including:
   an interface configured to receive the first signal, the second signal, and the third signal;
   a filter configured to allow passage of a signal having a frequency included in a specific frequency band; and
   a processor configured to calculate a concentration of a light absorber in blood of the subject based on the first signal and the second signal that have passed through the filter,
wherein a ratio of an extinction coefficient of a first light absorber in blood and an extinction coefficient of a second light absorber in blood is different between the first wavelength and the second wavelength;
wherein any one of the first light absorber in blood and the second light absorber in blood is the light absorber in blood;
wherein the third signal has a larger amplitude than the first signal and the second signal; and
wherein the processor is configured to determine the frequency band based on a frequency of the third signal.

An illustrative aspect of the present disclosure may provide a non-transitory computer-readable medium having stored a computer program adapted to be executed by a processor installed in a signal processing device, the computer program being configured to cause, when executed, the signal processing device to:
receive a first signal corresponding to an intensity of first light that has passed through a tissue of a subject, the first light including a first wavelength;
receive a second signal corresponding to an intensity of second light that has passed through the tissue, the second light including a second wavelength;
receive a third signal corresponding to an intensity of third light that has passed through the tissue, the third light including a third wavelength;
calculate a concentration of a light absorber in blood of the subject based on the first signal and the second signal that have passed through a filter configured to allow passage of a signal having a frequency included in a specific frequency band; and
determine the frequency band based on a frequency of the third signal,
wherein a ratio of an extinction coefficient of a first light absorber in blood and an extinction coefficient of a second light absorber in blood is different between the first wavelength and the second wavelength;
wherein any one of the first light absorber in blood and the second light absorber in blood is the light absorber in blood; and
wherein the third signal has a larger amplitude than the first signal and the second signal.

An illustrative aspect of the present disclosure may provide a photometry method, comprising:
receiving a first signal corresponding to an intensity of first light that has passed through a tissue of a subject, the first light including a first wavelength;
receiving a second signal corresponding to an intensity of second light that has passed through the tissue, the second light including a second wavelength;
receiving a third signal corresponding to an intensity of third light that has passed through the tissue, the third light including a third wavelength;
calculating a concentration of a light absorber in blood of the subject based on the first signal and the second signal that have passed through a filter configured to allow passage of a signal having a frequency included in a specific frequency band; and
determining the frequency band based on a frequency of the third signal,
wherein the first wavelength and the second wavelength are determined such that a ratio of an extinction coefficient of a first light absorber in blood and an extinction coefficient of a second light absorber in blood is made different therebetween;
wherein any one of the first light absorber in blood and the second light absorber in blood is the light absorber in blood; and
wherein the third wavelength is determined such that the third signal has a larger amplitude than the first signal and the second signal.

In each of the illustrative aspects described above, attention is paid to the fact that the first signal, the second signal, and the third signal have substantially similar shapes, so that the passband frequency range of the filter is determined based on the frequency of the third signal having the largest amplitude. For example, by determining the passband frequency range so as to include the frequency of the third signal that is substantially equal to the pulse wave frequency, the amplitude of the pulse wave signal is based on the light with the wavelength having a larger amplitude. Accordingly, artifact components can be efficiently removed from the first signal and the second signal that tend to have a lower S/N ratio. As a result, the measured value of the light absorber concentration in blood can be calculated based on the first signal and the second signal in which the S/N ratio is increased with respect to the pulse wave frequency component. Therefore, it is possible to suppress a decrease in calculation accuracy of the light absorber concentration in blood even in a case where the amplitude of the pulse wave signal to be obtained is small. This advantage is particularly remarkable in a case where the light absorber concentration in blood is measured for a subject for which the pulse wave signal to be obtained has a smaller amplitude (e.g., a hypoperfusion patient).

### DESCRIPTION OF THE DRAWINGS

FIG. 1 illustrates a functional configuration of a pulse oximeter according to an exemplary embodiment.

FIG. 2 illustrates pulse wave signals prior to passage of a filter of FIG. 1.

FIG. 3 illustrates pulse wave signals after the passage of the filter of FIG. 1.

### DESCRIPTION OF EMBODIMENTS

As used herein, the indefinite articles “a” and “an” are intended to mean “at least one” unless the context clearly indicates otherwise.

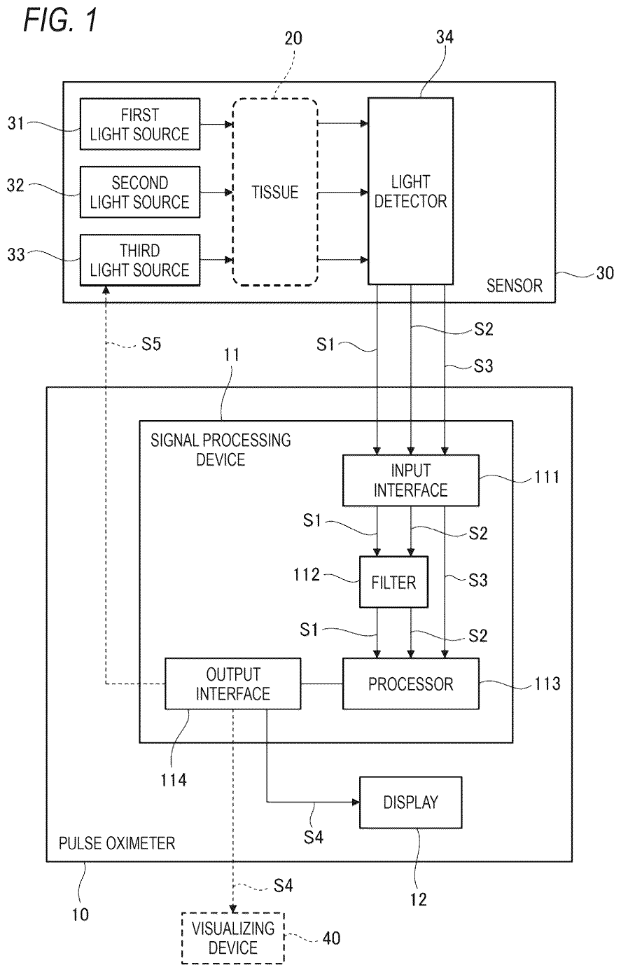

Exemplary embodiments are described in detail below with reference to the accompanying drawings. FIG. 1 illustrates a functional configuration of a pulse oximeter 10 according to an exemplary embodiment. The pulse oximeter 10 is a device configured to measure a transcutaneous arterial oxygen saturation (SpO2) of a subject based on a signal outputted from a sensor 30 attached to a tissue 20 of the subject. The SpO2 indicates a ratio of oxyhemoglobin to an amount of oxygen-carriable hemoglobin. The oxyhemoglobin is an exemplary light absorber in blood. The SpO2 is an exemplary light absorber concentration in blood. The pulse oximeter 10 is an exemplary medical photometer.

The sensor 30 includes a first light source 31, a second light source 32, a third light source 33, and a light detector 34. Each of the first light source 31, the second light source 32, and the third light source 33 is disposed so as to face the light detector 34 across the tissue 20.

The first light source 31 is configured to emit first light including a first wavelength λ1. In this example, the first wavelength λ1 is included in the infrared region. Examples of the first wavelength λ1 include 880 nm and 940 nm.

The second light source 32 is configured to emit second light including a second wavelength λ2. In this example, the second wavelength λ2 is included in the red region. Examples of the second wavelength λ2 include 630 nm and 660 nm.

The first wavelength λ1 and the second wavelength λ2 are determined such that a ratio of extinction coefficients of the first light absorber and the second light absorber is made different between these wavelengths. In the case of SpO2, the first wavelength λ1 and the second wavelength λ2 are determined such that a ratio of extinction coefficients of oxyhemoglobin and deoxyhemoglobin is made substantially different between these wavelengths.

In other words, the oxyhemoglobin is an exemplary first light absorber in blood, and the deoxyhemoglobin is an exemplary second light absorber in blood. Other exemplary light absorbers in blood that may be selected as a combination of the first light absorber in blood and the second light absorber in blood include carboxyhemoglobin and methemoglobin.

The third light source 33 is configured to emit third light including the third wavelength λ3. In this example, the third wavelength λ3 is included in the yellow region. Examples of the third wavelength λ3 include a wavelength included in a range of 570 nm to 610 nm.

Each of the first light source 31, the second light source 32, and the third light source 33 includes a semiconductor light emitter capable of emitting light with a predetermined wavelength. Examples of the semiconductor light emitter include a light emitting diode (LED), a laser diode, and an EL element.

The photo detector 34 includes an optical sensor having a sensitivity to each of the first wavelength λ1, the second wavelength λ2, and the third wavelength λ3. Examples of the optical sensor include a photodiode, a phototransistor, and a photoresistor.

The photo detector 34 is configured to output a first signal S1 corresponding to an intensity I1 of the first light that has passed through the tissue 20 of the subject. Similarly, the photo detector 34 is configured to output a second signal S2 and a third signal S3 respectively corresponding to an intensity I2 of the second light and an intensity I3 of the third light that have passed through the tissue 20.

Each of the first signal S1, the second signal S2, and the third signal S3 may be an analog signal or a digital signal in accordance with the specification of the photo detector 34.

The pulse oximeter 10 includes a signal processing device 11. The signal processing device 11 is a device configured to process the signals outputted from the sensor 30. The signal processing device 11 includes an input interface 111, a filter 112, a processor 113, and an output interface 114.

The input interface 111 is configured as a hardware interface adapted to receive the first signal S1, the second signal S2, and the third signal S3. In the case where each of the first signal S1, the second signal S2, and the third signal S3 is an analog signal, the input interface 111 is provided with an adequate conversion circuit including an A/D converter.

The filter 112 is configured to pass a signal in a specific frequency band. The filter 112 may be implemented by a well-known filter circuit.

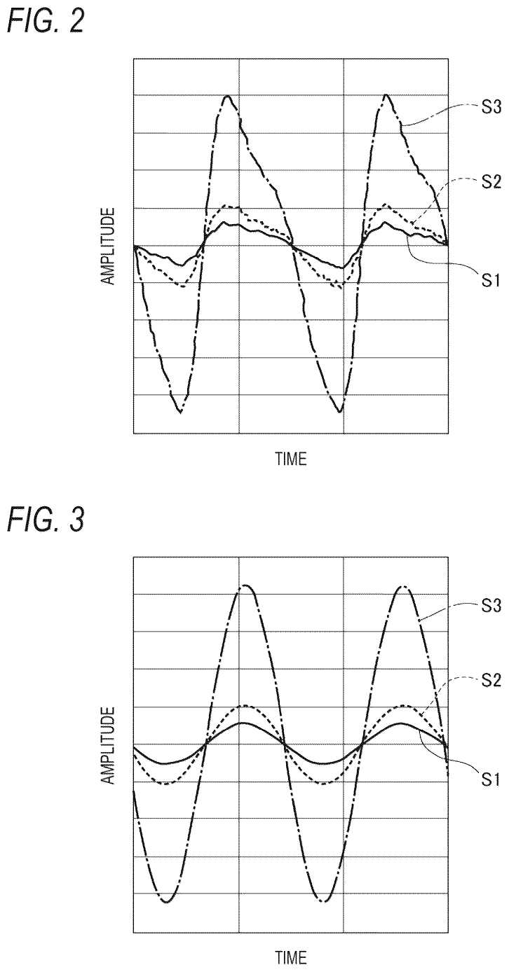

FIG. 2 illustrates the first signal S1, the second signal S2, and the third signal S3 before they are inputted to the filter 112. A quantity of light of each wavelength that has passed through the tissue 20 of the subject varies in accordance with blood pulsations of the subject. Accordingly, each of the first signal S1, the second signal S2, and the third signal S3 is obtained as a pulse wave signal in which an amplitude is varied at a frequency that is substantially equal to the pulsations. In the following description, this frequency is referred to as a “pulse wave frequency”.

As illustrated in FIG. 2, the amplitude of the third signal S3 is larger than the amplitudes of the first signal S1 and the second signal S2. In other words, the third wavelength λ3 is determined as a wavelength at which the third signal S3 having an amplitude larger than amplitudes of the first signal S1 and the second signal S2 can be obtained.

On the other hand, light with wavelengths having larger amplitudes are absorbed more strongly by the tissue 20. Accordingly, the light with such wavelengths encounter greater difficulty in passing through the tissue 20. The third wavelength λ3 is determined so as to satisfy the above-described requirement as for the amplitudes within a wavelength range capable of passing through the tissue 20. Alternatively, a thickness dimension of the tissue 20 is so determined as to allow passage of the third light. Exemplary body regions having such a tissue 20 include an earlobe and a nasal ala. In the case of a neonate, a fingertip may also be such an exemplary body region.

The processor 113 is configured to determine a frequency band allowed by the filter 112 to pass through based on a frequency of the third signal S3 corresponding to the intensity of the third light that has passed through the tissue 20. In other words, the passband frequency range of the filter 112 is variable. Specifically, the processor 113 identifies a pulse wave frequency based on the third signal S3, and determines the passband frequency range of the filter 112 so as to allow passage of signals with the pulse wave frequency.

FIG. 3 illustrates the first signal S1, the second signal S2, and the third signal S3 that have passed through the filter 112 that is so configured as to allow passage of a frequency band including the pulse wave frequency. It is apparent that artifact components included in the original signal have been removed. It should be noted that the third signal S3 need not be supplied to the filter 112.

The processor 113 is configured to calculate an SpO2 of the subject based on the first signal S1 and the second signal S2 that have passed through the filter 112. Hereinafter, a specific flow of the calculation process will be described.

Based on the temporal variations of the first signal S1, the processor 113 obtains a variation ΔA1 of the light attenuation of the first light due to the blood pulsations of the subject. The variation ΔA1 of the light attenuation is expressed as follows.

$$Δ A 1 = ln [ S 1 / ( S 1 - Δ S 1 ) ] \approx Δ S 1 / S 1 \qquad ( 1 )$$

Here, ΔS1 indicates a variation of the first signal S1 due to the blood pulsations of the subject.

Similarly, the processor 113 obtains a variation ΔA2 of light attenuation of the second light due to the blood pulsations of the subject, based on the temporal variations of the second signal S2. The variation ΔA2 of the light attenuation is expressed as follows.

$$Δ A 2 = ln [ S 2 / ( S 2 - Δ S 2 ) ] \approx Δ S 2 / S 2 \qquad ( 2 )$$

Here, ΔS2 indicates a variation of the second signal S2 due to the blood pulsations of the subject.

The processor 113 is configured to calculate a measured value S of the subject's SpO2 based on the variation ΔA1 and the variation ΔA2. Specifically, it is configured to execute the following processing.

The variation ΔA1 and the variation ΔA2 can be respectively expressed as follows. Here, E denotes an extinction coefficient (dl g⁻¹ cm⁻¹). Hb denotes a blood hemoglobin concentration (g dl⁻¹). Σ denotes the light attenuation rate (cm⁻¹). ΔD denotes a thickness variation (cm) due to the blood pulsations. The index “b” denotes blood. The index “t” denotes a tissue other than the blood. The index “1” denotes the first light. The index “2” denotes the second light.

$$Δ A 1 = Δ Ab 1 + Δ At 1 = Eb 1 \cdot Hb \cdot Δ Db + \sum t 1 \cdot Δ Dt \qquad ( 3 )$$

$$Δ A 2 = Δ Ab 2 + Δ At 2 = Eb 2 \cdot Hb \cdot Δ Db + \sum t 2 \cdot Δ Dt \qquad ( 4 )$$

Equations (3) and (4) can be modified as follows.

$$\begin{matrix}Δ A 1 = Eb 1 \cdot Hb \cdot Δ Db + \sum t 1 \cdot Δ Dt \\ = [ Eb 1 + ( \sum t 1 \cdot Δ Dt ) / ( Hb \cdot Δ Db ) ] \cdot ( Hb \cdot Δ Db ) \\ = ( Eb 1 + Ex 1 ) \cdot ( Hb \cdot Δ Db )\end{matrix} \qquad ( 5 )$$

$$\begin{matrix}Δ A 2 = Eb 2 \cdot Hb \cdot Δ Db + \sum t 2 \cdot Δ Dt \\ = [ Eb 2 + ( \sum t 2 \cdot Δ Dt ) / ( Hb \cdot Δ Db ) ] \cdot ( Hb \cdot Δ Db ) \\ = ( Eb 2 + Ex 2 ) \cdot ( Hb \cdot Δ Db )\end{matrix} \qquad ( 6 )$$

Here, Ex is a variable for which (Σt·ΔDt)/(Hb·ΔDb) is replaced. The index “1” denotes the first light. The index “2” denotes the second light.

Equations (5) and (6) can be modified as follows.

$$Eb 1 + Ex 1 - [ Δ A 1 / ( Hb \cdot Δ Db ) ] = 0 \qquad ( 7 )$$

$$Eb 2 + Ex 2 - [ Δ A 2 / ( Hb \cdot Δ Db ) ] = 0 \qquad ( 8 )$$

As for Equation (8), the blood extinction coefficient Eb2 for the second light can be approximated by the blood extinction coefficient Eb1 for the first light as follows.

$$Eb 2 = a 2 \cdot Eb 1 + b2 \qquad ( 9 )$$

Here, “a” and “b” are constants. The index “1” denotes the first light. The index “2” denotes the second light.

The Ex2 for the second light may be approximated by the Ex1 for the first light as follows.

$$Ex 2 = α 2 \cdot Ex 1 + β 2 \qquad ( 10 )$$

Here, “α” and “β” are constants. The index “1” denotes the first light. The index “2” denotes the second light.

Equations (7) and (8) can be rewritten using Equations (9) and (10), yielding the following equations.

$$Eb 1 + Ex 1 - [ Δ A 1 / ( Hb \cdot Δ Db ) ] = 0 \qquad ( 11 )$$

$$EB 1 - [ Δ A 1 / ( Hb \cdot Δ Db ) ] = - Ex 1$$

$$( a 2 \cdot Eb 1 + b 2 ) + ( α 2 \cdot Ex 1 + β 2 ) - [ Δ A 2 / ( Hb \cdot Δ Db ) ] = 0 \qquad ( 12 )$$

$$a 2 \cdot Eb 1 - [ Δ A 2 / ( Hb \cdot Δ Db ) ] = - α 2 \cdot Ex 1 - β 2 - b 2$$

Using a statistically derived constant for Ex1, a determinant below yields values of variables Eb1 and HbΔDb.

$$( \begin{matrix}1 & - Δ A 1 \\ a 2 & - Δ A 2\end{matrix} ) ( \begin{matrix}Eb 1 \\ \frac{1}{Hb \cdot Δ Db}\end{matrix} ) = ( \begin{matrix}- Ex 1 \\ - α 2 \cdot Ex 1 - β 2 - b 2\end{matrix} ) \qquad ( 13 )$$

Converting SpO2 from percentage notation to S in decimal form, the blood extinction coefficient Eb1 for the first light can be expressed as follows.

$$Eb 1 = Eo 1 \cdot S + Er 1 ( 1 - S ) \qquad ( 14 )$$

Here, Eo denotes an extinction coefficient of oxyhemoglobin. Er denotes an extinction coefficient of deoxyhemoglobin. The index “1” denotes the first light. Accordingly, the processor 113 calculates the measured value S of SpO2 with the following equation.

$$S = ( Eb 1 - Er 1 ) / ( Eo 1 - Er 1 ) \qquad ( 15 )$$

As illustrated in FIG. 1, the pulse oximeter 10 includes a display 12. The processor 113 of the signal processing device 11 is configured to output, from the output interface 114, a control signal S4 that causes the display 12 to display the calculated measured value S of SpO2. The control signal S4 may be an analog signal or a digital signal in accordance with the specification of the display 12.

In other words, the output interface 114 is configured as a hardware interface adapted to output the control signal S4. In the case where the control signal S4 is an analog signal, the output interface 114 is provided with an adequate conversion circuit including a D/A converter. This description is similarly applied to other signals (described later) that can be outputted by the output interface 114.

In this exemplary embodiment, attention is paid to the fact that the first signal S1, the second signal S2, and the third signal S3 have substantially similar shapes, so that the passband frequency range of the filter 112 is determined based on the frequency of the third signal S3 having the largest amplitude. For example, by determining the passband frequency range so as to include the frequency of the third signal S3 that is substantially equal to the pulse wave frequency, the amplitude of the pulse wave signal is based on the light with the wavelength having a larger amplitude. Accordingly, artifact components can be efficiently removed from the first signal S1 and the second signal S2 that tend to have a lower S/N ratio. As a result, the measured value of SpO2 can be calculated based on the first signal S1 and the second signal S2 in which the S/N ratio is increased with respect to the pulse wave frequency component. Therefore, it is possible to suppress a decrease in calculation accuracy of SpO2 even in a case where the amplitude of the pulse wave signal to be obtained is small. This advantage is particularly remarkable in a case where SpO2 is measured for a subject for which the pulse wave signal to be obtained has a smaller amplitude (e.g., a hypoperfusion patient).

The third light that is used to determine the passband frequency range of the filter 112 need not always be incident on the tissue 20. As illustrated in FIG. 1, the processor 113 of the signal processing device 11 is configured to output, from the output interface 114, an emission control signal S5 that causes the third light source 33 to emit the third light in a case where at least one of the first signal S1 and the second signal S2 has an amplitude less than a threshold. The emission control signal S5 may be an analog signal or a digital signal in accordance with the specification of the third light source 33.

According to the above configuration, the third light is emitted in a case where it is difficult to perform measurement of SpO2 with required accuracy because of the fact that the amplitude of at least one of the first signal S1 and the second signal S2 is small, so that the above-described processing for increasing the S/N ratio of the first signal S1 and the second signal S2 is executed. Accordingly, it is possible to suppress a decrease in the calculation accuracy of SpO2 while suppressing the power consumption by the sensor 30.

The processor 113 of the signal processing device 11 having various functions described above may be implemented by at least one versatile microprocessor configured to cooperate with at least one versatile memory. Examples of the versatile microprocessor include a CPU, an MPU, and a GPU. Examples of the versatile memory include a ROM and a RAM. In this case, a computer program that implements the various functions described above may be stored in the ROM. The ROM is an exemplary non-transitory computer-readable medium having stored a computer program. The versatile microprocessor designates at least a part of the program stored in the ROM, loads the designated program in the RAM, and executes the above-described processing in cooperation with the RAM. The computer program may be pre-installed in a versatile memory, or may be downloaded from an external server device with a communication network, and then installed in the versatile memory. In this case, the external server device is an exemplary non-transitory computer-readable medium having stored a computer program.

The processor 113 may be implemented by at least one exclusive integrated circuitry capable of executing the above-described computer program. Examples of the exclusive integrated circuitry include a microcontroller, an ASIC, and an FPGA. In this case, the above-described computer program is pre-installed in the memory element included in the exclusive integrated circuitry. The memory element is an exemplary non-transitory computer-readable medium having stored a computer program. The processor 113 may also be implemented by a combination of a versatile microprocessor and an exclusive integrated circuitry.

The various configurations described above are merely illustrative for facilitating understanding of the present disclosure. Each of the illustrative configurations may be appropriately modified or combined with another illustrative configuration within the scope of the present disclosure.

In the above exemplary embodiment, the first signal S1, the second signal S2, and the third signal S3 outputted from the sensor 30 attached to the subject are directly inputted to the signal processing device 11. However, the signal processing device 11 may be disposed at a location remote from the sensor 30. In this case, the first signal S1, the second signal S2, and the third signal S3 are transmitted to the signal processing device 11 via a wired or wireless communication network.

In the above exemplary embodiment, the display 12 on which the calculated measured value S of SpO2 is displayed is provided as a part of the pulse oximeter 10 together with the signal processing device 11. However, the measured value S may be visualized by a visualization device 40 that is located at a position remote from the signal processing device 11. Examples of the visualization device include a display that displays the measured value S, a projector that projects the measured value S, and a printer that prints the measured value S. In this case, the control signal S4 outputted from the output interface 114 of the signal processing device 11 is transmitted to the visualization device 40 via the wired or wireless communication network.

Accordingly, the signal processing device 11 need not be installed in the pulse oximeter 10 as long as the first signal S1, the second signal S2, and the third signal S3 can be received from the sensor 30, and the calculated measured value S of SpO2 can be visualized in the visualization device 40.

1. A signal processing device, comprising:
an interface configured to receive:
a first signal corresponding to an intensity of first light that has passed through a tissue of a subject, the first light including a first wavelength;
a second signal corresponding to an intensity of second light that has passed through the tissue, the second light including a second wavelength; and
a third signal corresponding to an intensity of third light that has passed through the tissue, the third light including a third wavelength;
a filter configured to allow passage of a signal having a frequency included in a specific frequency band; and
a processor configured to calculate a concentration of a light absorber in blood of the subject based on the first signal and the second signal that have passed through the filter,
wherein a ratio of an extinction coefficient of a first light absorber in blood and an extinction coefficient of a second light absorber in blood is different between the first wavelength and the second wavelength;
wherein any one of the first light absorber in blood and the second light absorber in blood is the light absorber in blood;
wherein the third signal has a larger amplitude than the first signal and the second signal; and
wherein the processor is configured to determine the frequency band based on a frequency of the third signal.

2. The signal processing device according to claim 1,
wherein the processor is configured to output a control signal that causes a light source to emit the third light in a case where an amplitude of at least one of the first signal and the second signal is less than a threshold.

3. The processing device according to claim 1,
wherein the first wavelength is included in an infrared region;
wherein the second wavelength is included in a red region; and
wherein the third wavelength is included in a yellow region.

4. The processing device according to claim 1,
wherein the concentration of the light absorber in blood is a transcutaneous arterial oxygen saturation.

5. A medical photometer, comprising:
a sensor configured to output:
a first signal corresponding to an intensity of first light that has passed through a tissue of a subject, the first light including a first wavelength;
a second signal corresponding to an intensity of second light that has passed through the tissue, the second light including a second wavelength; and
a third signal corresponding to an intensity of third light that has passed through the tissue, the third light including a third wavelength; and
a signal processing device including:
an interface configured to receive the first signal, the second signal, and the third signal;
a filter configured to allow passage of a signal having a frequency included in a specific frequency band; and
a processor configured to calculate a concentration of a light absorber in blood of the subject based on the first signal and the second signal that have passed through the filter,
wherein a ratio of an extinction coefficient of a first light absorber in blood and an extinction coefficient of a second light absorber in blood is different between the first wavelength and the second wavelength;
wherein any one of the first light absorber in blood and the second light absorber in blood is the light absorber in blood;
wherein the third signal has a larger amplitude than the first signal and the second signal; and
wherein the processor is configured to determine the frequency band based on a frequency of the third signal.

6. The medical photometer according to claim 5,
wherein the sensor includes:
a first light source configured to emit the first light;
a second light source configured to emit the second light; and
a third light source configured to emit the third light; and
a light detector configured to receive the first light, the second light, and the third light that have passed through the tissue,
wherein the first light source, the second light source, and the third light source are arranged so as to face the light detector across the tissue.

7. A non-transitory computer-readable medium having stored a computer program adapted to be executed by a processor installed in a signal processing device, the computer program being configured to cause, when executed, the signal processing device to:
receive a first signal corresponding to an intensity of first light that has passed through a tissue of a subject, the first light including a first wavelength;
receive a second signal corresponding to an intensity of second light that has passed through the tissue, the second light including a second wavelength;
receive a third signal corresponding to an intensity of third light that has passed through the tissue, the third light including a third wavelength;
calculate a concentration of a light absorber in blood of the subject based on the first signal and the second signal that have passed through a filter configured to allow passage of a signal having a frequency included in a specific frequency band; and
determine the frequency band based on a frequency of the third signal,
wherein a ratio of an extinction coefficient of a first light absorber in blood and an extinction coefficient of a second light absorber in blood is different between the first wavelength and the second wavelength;
wherein any one of the first light absorber in blood and the second light absorber in blood is the light absorber in blood; and
wherein the third signal has a larger amplitude than the first signal and the second signal.

8. A photometry method, comprising:
receiving a first signal corresponding to an intensity of first light that has passed through a tissue of a subject, the first light including a first wavelength;
receiving a second signal corresponding to an intensity of second light that has passed through the tissue, the second light including a second wavelength;
receiving a third signal corresponding to an intensity of third light that has passed through the tissue, the third light including a third wavelength;
calculating a concentration of a light absorber in blood of the subject based on the first signal and the second signal that have passed through a filter configured to allow passage of a signal having a frequency included in a specific frequency band; and
determining the frequency band based on a frequency of the third signal,
wherein the first wavelength and the second wavelength are determined such that a ratio of an extinction coefficient of a first light absorber in blood and an extinction coefficient of a second light absorber in blood is made different therebetween;
wherein any one of the first light absorber in blood and the second light absorber in blood is the light absorber in blood; and
wherein the third wavelength is determined such that the third signal has a larger amplitude than the first signal and the second signal.
